(19) United States
(12) Patent Application Publication
(10) Pub. No.: US 2007/0066868 A1
(43) Pub. Date: Mar. 22, 2007

(54) CAPSULE ENDOSCOPE

(75) Inventor: Shinichi Shikii, Kangawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD., Kanagawa (JP)

(21) Appl. No.: 11/534,101

(22) Filed: Sep. 21, 2006

(30) Foreign Application Priority Data
Sep. 21, 2005 (JP) 2005-273537

(51) Int. Cl. A61B 1/04; A61B 1/06

(52) U.S. Cl. 600/118; 600/160

(57) ABSTRACT

A capsule endoscope is provided with an image pickup section having a CCD image sensor, a plurality of LEDs for emitting illumination light, a control circuit for controlling the image pickup section and the LEDs, a transmitting/receiving circuit for transmitting an image signal as a radio signal, and a battery for supplying electric power to each part of the capsule endoscope inside of a capsule case. When the capsule endoscope reaches a large intestine which is a lower digestive tract, the transmitting/receiving circuit receives an image capture initiation signal from an external transmitter provided outside of the body. The control circuit then activates the LEDs and the image pickup section to perform the image capturing at a predetermined interval.

### BACKGROUND OF THE INVENTION

1. Field of the Invention

The present invention relates to a capsule endoscope, in which an image pickup section is contained in a small swallowable capsule case, for capturing images inside of a digestive tract in a living body.

2. Description Related to the Prior Art

In medical diagnosis by a conventional endoscope system, an endoscope having an operation section and an insertion section is used. The insertion section is a long tube incorporating a CCD camera at its front end, and is inserted through a patient's mouth to capture images inside of the digestive tract. The captured images are observed on a monitor device. In the conventional endoscope system, some disadvantages are pointed out, such as an enormous physical strain on the patient due to the insertion of the insertion section into the body, difficulty of the inspection of a small intestine and a large intestine which reside far away from the mouth. In view of this, a swallowable capsule endoscope system has been proposed in recent years (for example, see International Publication No. WO 03/009739 corresponding to (Kohyo) National Publication of Translated Version No. 2004-535878).

The capsule endoscope has a capsule case and contains a compact image sensor, a light source like light emitting diodes (LEDs), a transmitter for transmitting an image signal as radio waves and a battery in the capsule case. In the same manner as foods are ingested, the capsule endoscope is taken into the body through the mouth and reaches an esophagus, a stomach and the like. In this way, after slowly moving through the digestive tract (small intestine, large intestine) owing to a peristaltic movement thereof, the capsule endoscope is passed out of the body. The capsule endoscope captures images inside the body at a rate of, for example, 30 frames/sec while moving through the body and transmits the image signals as the radio waves to a receiver outside of the body. The patient has only to swallow the capsule and put some external receivers for receiving the image signals on the body surface, therefore the physical stain is reduced. In addition, the capsule endoscope is capable of capturing images of the digestive tract in every corner, and thus early detection of disease may be expected.

However, the capsule endoscope requires relatively large electricity for a single observation since the LEDs illuminate a region to be inspected every time the image is captured. For the image capturing of the digestive tract, the capsule endoscope stays therein for a long time. In order to support the image capturing throughout this stay, the battery needs to have a large capacity. This makes it difficult to miniaturize the capsule endoscope. Moreover, some of the battery power is wasted when only a lower digestive tract (for example, large intestine) is needed to be captured.

### SUMMARY OF THE INVENTION

It is a main object of the present invention to provide a capsule endoscope miniaturized by using a battery efficiently.

It is another object of the present invention to provide a capsule endoscope capable of reliably capturing images of a lower digestive tract through which the capsule endoscope passes.

In order to achieve the above and other objects, a capsule endoscope of the present invention includes an image pickup section for capturing an image of a region to be inspected to generate an image signal; a light source section for emitting illumination light every time when the image pickup section captures the image; an image transmitter for transmitting the image signal to an external receiver via radio waves; a battery for supplying electric power to each of the image pickup section, the light source section and the image transmitter; an image capture controller; and a capsule case for containing the image pickup section, the light source section, the image transmitter, the image capture controller and the battery. The image capture controller has a power saving mode in which an image capturing rate per unit time is low and a normal capture mode in which the image capture rate per unit time is high. The image capture controller selects one of these modes to execute.

The image capture controller selects the power saving mode in an initial state and selects the normal capture mode while the capsule endoscope moves inside of a body. It is preferable that the image capturing is not performed in the power saving mode.

Preferably, the capsule endoscope further includes a receiver for receiving a mode changeover signal from an external transmitter. The image capture controller selects the normal capture mode when the receiver receives the mode changeover signal.

Preferably, the capsule endoscope further includes a pressure detecting section for detecting pressure from outside of the capsule case and a timer for counting time. The image capture controller selects the normal capture mode when the pressure detected by the pressure detecting section changes from a standard value. The standard value is a pressure value at a time when the timer counts a predetermined amount of time.

Preferably, the capsule endoscope further includes an image analyzing section for analyzing a captured image in order to identify a predetermined region to be inspected. The image capture controller selects the normal capture mode when the image analyzing section identifies the image captured in the power saving mode as an image of the predetermined region to be inspected.

According to the present invention, the normal capture mode in which the image capturing rate is high and the power saving mode in which the image capturing rate is low and thereby saving the battery are selectable. Therefore, unnecessary image capturing is not performed and it is possible to reliably capture the images of the region to be inspected. Owing to this, the battery can be miniaturized, and furthermore, the capsule endoscope itself can be miniaturized. When the peristalsis of the digestive tract is slow, it takes time until the capsule endoscope reaches the region to be inspected, such as the large intestine. According to the present invention, the battery is used effectively by switching between the normal capture mode and the power saving mode, therefore it is prevented that the battery runs down while capturing the images of the region to be inspected and ends up with not being able to capture necessary images. Owing to this, the patient does not have to swallow the capsule endoscope again.

The power saving mode is selected in the initial state and the normal capture mode is selected while the capsule endoscope moves inside of the body. Owing to this, the images of the lower digestive tract, such as the large intestine or a distal portion of the small intestine, can be captured even with the small battery having small capacity.

By not performing the image capturing in the power saving mode, the battery can be further miniaturized. In addition, the battery can be saved in the power saving mode and therefore the output of the radio waves from the capsule endoscope can be higher. Owing to this, for example, the number of the external receivers for receiving the radio waves of the image signal can be reduced. Accordingly, the burden of the inspection for the patient who has to put the external receivers on the body surface for long time can be reduced.

In addition, when an effective area of the radio waves of the mode changeover signal is made small, it is possible to start the image capturing of the region to be inspected at a more accurate position. For example, when the large intestine is needed to be captured, the external transmitter is placed at right lower abdominal quadrant. By placing the external transmitter at this position, the image capturing starts when the capsule endoscope moves from the small intestine to the large intestine, and therefore the unnecessary image capturing before the large intestine is not performed.

The capsule endoscope is provided with the timer and the pressure detecting section for detecting the pressure from outside of the capsule case. For this configuration, it is possible to firstly count the presumed time from when the capsule endoscope is orally taken into the body till when the capsule endoscope reaches a particular digestive tract, and then detect the pressure on the capsule endoscope changing due to the difference of each digestive tract's inner diameter. For example, the presumed time till when the capsule endoscope reaches the small intestine is counted by the timer. After the count, the pressure change is detected to recognize that the capsule endoscope moves from the small intestine to the large intestine. Thereby, the image capturing can be started at an appropriate position. For this configuration, only the entire area of the large intestine is accurately captured.

The image analyzing section analyzes the image captured in the power saving mode in which the image capturing rate is low. When the captured image is identified as that of the region to be inspected, that is, the region to be captured in detail, the normal capture mode is selected to increase the image capturing rate. Owing to this, necessary images can be obtained with minimal battery power.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a block schematic diagram illustrating a capsule endoscope according to a first embodiment;

FIG. 2 is an explanatory view illustrating a condition in which external receivers and an external transmitter are attached to a human body surface;

FIG. 3 is a flowchart illustrating an operation process of the capsule endoscope according to the first embodiment;

FIG. 4 is a block schematic diagram illustrating the capsule endoscope according to a second embodiment;

FIG. 5 is a flowchart illustrating the operation process of the capsule endoscope according to the second embodiment;

FIG. 6 is a block schematic diagram illustrating the capsule endoscope according to a third embodiment; and

FIG. 7 is a flowchart illustrating the operation process of the capsule endoscope according to the third embodiment.

### DESCRIPTION OF THE PREFERRED EMBODIMENTS

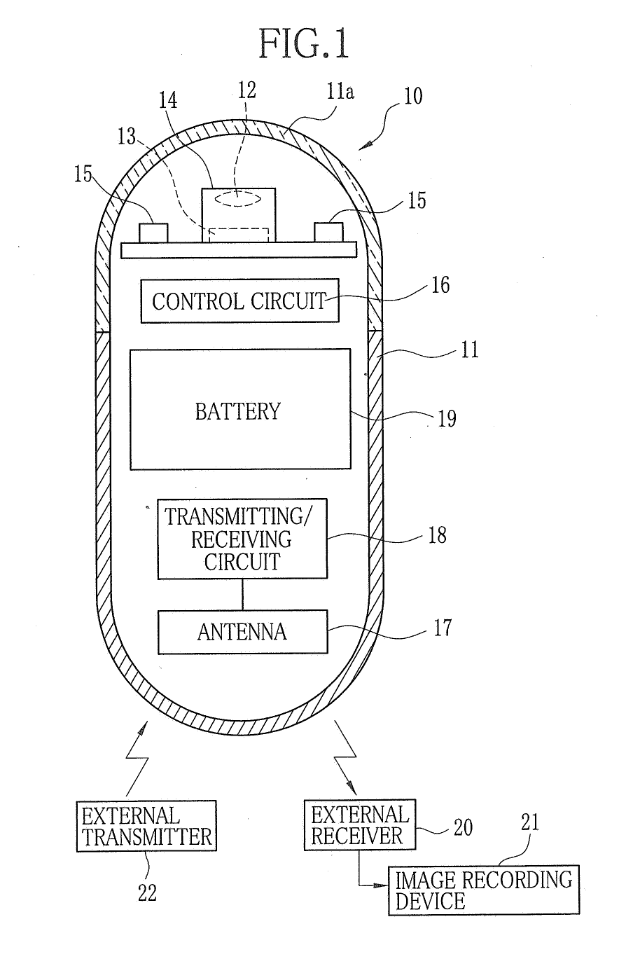

In FIG. 1, a capsule endoscope 10 has a capsule case 11 of a cylindrical shape with hemispheric ends. Inside of the capsule case 11 is provided with an image pickup section 14 having a lens 12 and a CCD image sensor 13, a plurality of LEDs 15 as an illumination light source, a control circuit 16 for controlling transmitting/receiving circuit 18 having an antenna 17, and a battery 19 for supplying electric power to each part.

A cap 11a of the capsule case 11 facing the image pickup section 14 is transparent. Note that the whole capsule case 11 may be made transparent. The lens 12 focuses light entered from outside of the capsule case 11 to form an image on the CCD image sensor 13. The CCD image sensor 13 photoelectrically converts the image on a pixel to pixel basis and generates an electric image signal. The LEDs 15 are disposed in a circle about the image pickup section 14 and illuminates forward thereof.

The control circuit 16 controls each part of the capsule endoscope 10. The control circuit 16 also performs power management. That is, the control circuit 16 judges the necessity of the electric power for each part and supplies the electric power from the battery 19 only when necessary. The control circuit 16 further generates a clock signal for driving the CCD image sensor 13, and controls on/off of the CCD image sensor 13. In addition, the control circuit 16 determines illumination interval of the LEDs 15 so as to illuminate the LEDs 15 for a predetermined time in synchronization with the timing of the image capturing by the CCD image sensor 13.

The transmitting/receiving circuit 18 is provided with a radio wave generating circuit and transmits the image signal from the control circuit 16 as a radio signal through the antenna 17. The transmitted radio signal is received by an external receiver 20 provided outside of the body. The external receiver 20 sends the received image signal to an image recording device 21. The image recording device 21 stores the captured images. The transmitting/receiving circuit 18 also receives an image capture initiation signal from an external transmitter 22 and sends it to the control circuit 16.

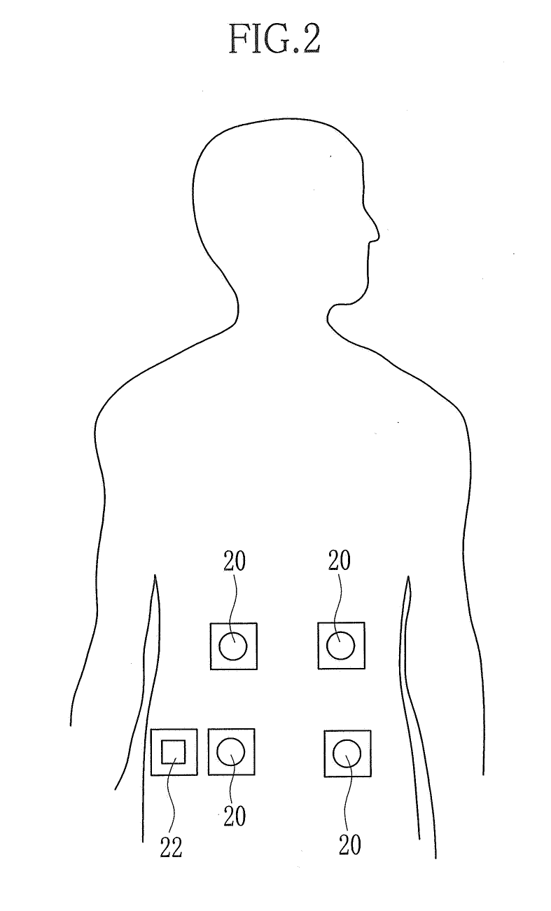

In FIG. 2, a plurality of the external receivers 20 are placed on an abdominal area and a back of a patient so that the radio signal of the image can be received anywhere. The external receiver 22 is placed at a position where the antenna 17 of the capsule endoscope 10 can receive the image capture initiation signal when the capsule endoscope 10 reaches a region to be inspected. In the present embodiment, the large intestine is the region to be inspected, and the external receiver 22 is placed at right lower abdominal quadrant of the patient's body. An effective area of the radio waves of the image capture initiation signal from the external transmitter 22 is, for example, 5 cm to 10 cm. Accordingly, the image capture initiation signal is not received by the antenna 17 until the capsule endoscope 10 reaches near an entrance of the large intestine.

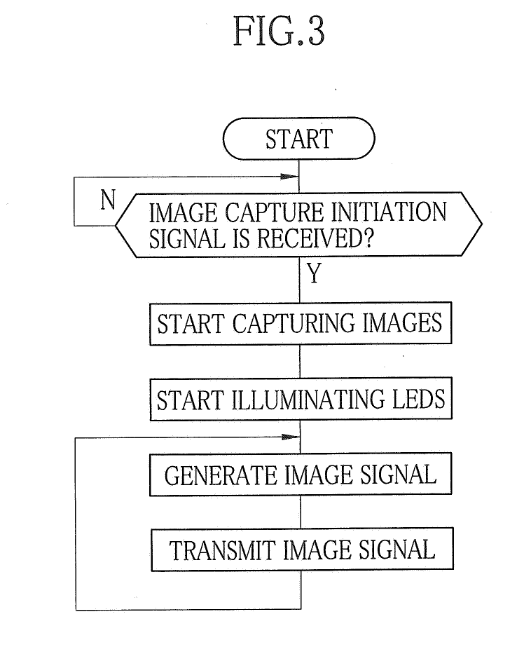

Next, an operation of the capsule endoscope 10 is explained. In FIG. 3, the capsule endoscope 10 is used for the patient who receives an inspection of the large intestine. In the capsule endoscope 10, the control circuit 16 stops the power supply to the CCD image sensor 13 and the LEDs 15. That is, the capsule endoscope 10 is in a power saving mode in which the image capturing and the emission of the illumination light are not performed. The capsule endoscope 10 is swallowed by the patient and reaches the esophagus and the stomach, and slowly passes through a duodenum and the small intestine. When the capsule endoscope 10 reaches near the entrance of the large intestine, the capsule endoscope 10 enters the effective area of the radio waves of the image capture initiation signal from the external transmitter 22, and the image capture initiation signal is received by the antenna 17.

The transmitting/receiving circuit 18 sends the received image capture initiation signal to the control circuit 16. Upon receiving the signal, the control circuit 16 switches from the power saving mode to a normal capture mode, thereby starting the power supply to the CCD image sensor 13 and the LEDs 15 and starts capturing images with the image pickup section 14. The LEDs 15 blinks at a regular interval and emits the illumination light in synchronization with the timing of the image capturing of the CCD image sensor 13. The illumination light emitted by the LEDs 15 illuminates inside of the digestive tract. The light reflected on the inside of the digestive tract enters the lens 12 of the image pickup section 14. The lens 12 forms the image of the inside of the digestive tract on an imaging surface of the CCD image sensor 13. The CCD image sensor 13 photoelectrically converts the image to generate the image signal.

The image signal from the CCD image sensor 13 is input to the control circuit 16. The control circuit 16 performs basic signal processing, such as amplification, digital conversion and the like over the image signal and sends the processed image signal to the transmitting/receiving circuit 18. The transmitting/receiving circuit 18 sends the image signal as the radio signal through the antenna 17. The transmitted image signal is received by the external receiver 20. The external receiver 20 sends the image signal to the image recording device 21. In this way, the captured images are stored in the image recording device 21.

While the capsule endoscope 10 moves through the digestive tract, the images thereof are captured at a regular frame rate. The image signal is received by the external receiver 20 close to the position of the capsule endoscope 10. Since the capsule endoscope 10 starts the image capturing after it reaches the large intestine, the battery power is not wasted. Accordingly, the battery 19 does not run down until the capsule endoscope 10 is passed out of the body and therefore necessary images of the region to be inspected are reliably captured. The captured images stored in the image recording device 21 are read out and displayed on a monitor device (not shown). Medical diagnosis is performed by observing the images on the monitor device.

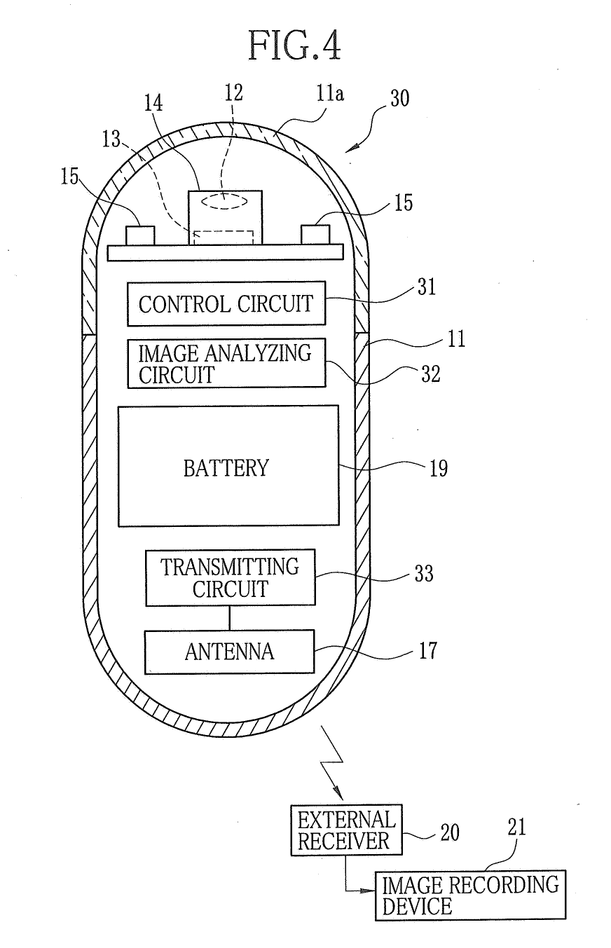

Next, a second embodiment of the present invention shown in FIG. 4 is explained. Note that the components identical to those of the first embodiment are denoted by the same reference numerals, and descriptions thereof are omitted. A capsule endoscope 30 has the image pickup section 14, the LEDs 15, a control circuit 31, an image analyzing circuit 32, a transmitting circuit 33 and the battery 19 contained in the capsule case 11. The control circuit 31 controls the operation of the capsule endoscope 30. The control circuit 31 is preliminary provided with a power saving mode in which the image capturing is performed at a low frame rate of 2 frames/sec, and a normal capture mode in which the image capturing is performed at a high frame rate of 30 frames/sec. The control circuit 31 selects and executes one of these modes.

Upon receiving the image signal from the CCD image sensor 13 through the control circuit 31, the image analyzing circuit 32 analyzes the image in order to identify a predetermined region to be inspected. The region to be inspected is, for example, the large intestine, and the captured image is judged whether it is the image of the large intestine or not by analyzing its features. The image signal from the CCD image sensor 13 is sent to the transmitting circuit 33 through the control circuit 31 and transmitted as the radio signal through the antenna 17. In the present embodiment, the capsule endoscope 30 is used for a patient who receives the inspection of the large intestine.

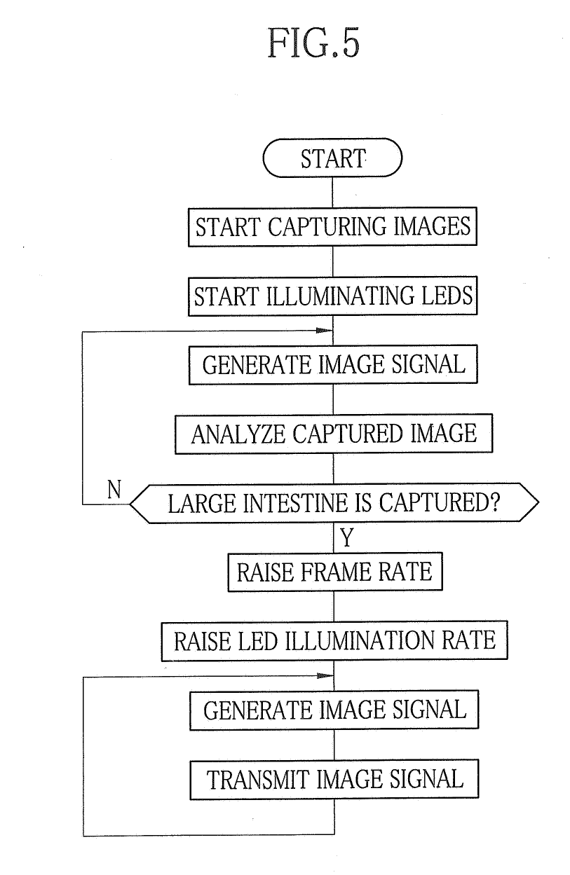

In FIG. 5, the capsule endoscope 30 normally executes the power saving mode, and the CCD image sensor 13 outputs the image signals at the frame rate of 2 frames/sec until the capsule endoscope 30 reaches the large intestine. The LEDs 15 emit the illumination light 2 times/sec in tandem with the image capturing operation of the CCD image sensor 13. At this time, the transmitting circuit 33 is not activated, therefore the radio signals of the images are not transmitted from the capsule endoscope 30. The CCD image sensor 13 captures images at the rate of 2 frames/sec and sends the image signals to the image analyzing circuit 32. The image analyzing circuit 32 judges every captured image for whether the large intestine appears or not, and the judgment results are kept in the control circuit 31.

The control circuit 31 continues the execution of the power saving mode until the image analyzing circuit 32 judges the captured image as that of the large intestine. When the image analyzing circuit 32 judges so, it sends the judgment signal which indicates that the capsule endoscope 30 reaches the large intestine to the control circuit 31. The control circuit 31 switches from the power saving mode to the normal capture mode. The control circuit 31 raises the frame rate from 2 frames/sec of the power saving mode to 30 frames/sec of the normal capture mode. At the same time, the control circuit 31 makes the LEDs 15 illuminate 30 times/sec to synchronize with the image capturing. The control circuit 31 stops the operation of the image analyzing circuit 32 to terminate the image analyzing process, and activates the transmitting circuit 33 to start the transmission of the image signals as the radio signals.

Until the capsule endoscope 30 is passed out of the body, the transmitting circuit 33 keeps transmitting the image signals at the rate of 30 frames/sec. The external receiver 20 sends the received image signals to the image recording device 21. The captured images are stored sequentially in the image recording device 21. In the image recording device 21, the images of the entire area of the large intestine are stored. The medical diagnosis is performed by using these images.

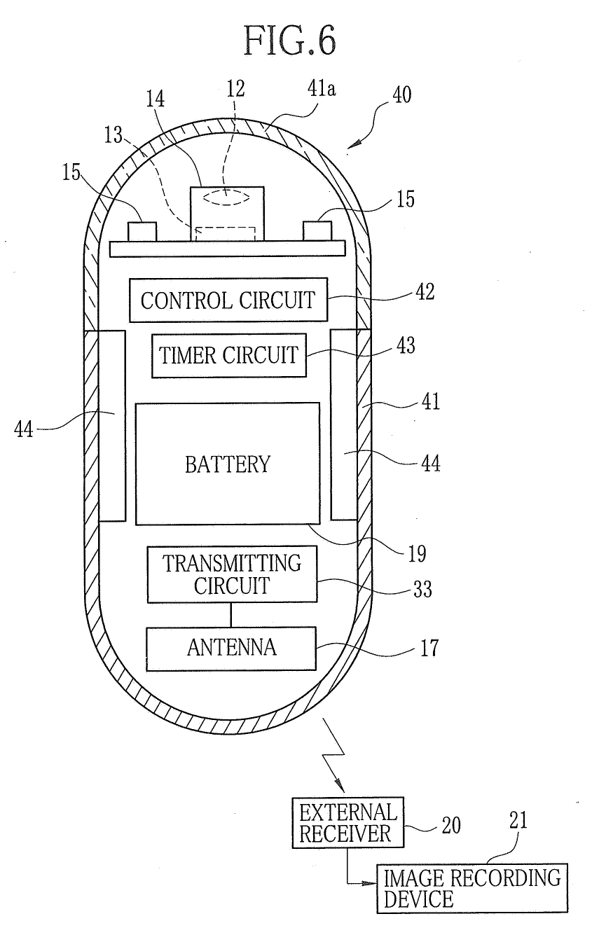

Next, a third embodiment of the present invention is explained with referring to FIG. 6. Note that the components identical to those of the first or second embodiment are denoted by the same reference numerals, and descriptions thereof are omitted. A capsule endoscope 40 has the image pickup section 14, the LEDs 15, a control circuit 42, a timer circuit 43, a pressure sensor like a piezoelectric sensor 44, the transmitting circuit 33 and the battery 19 contained in a capsule case 41. A cap 41a of the capsule case 41 facing the image pickup section 14 is transparent or the whole capsule case 41 may be made transparent. The capsule case 41 is elastically deformed in response to pressure from outside. The piezoelectric sensor 44 measures degree of the elastic deformation of the capsule case 41 and generates voltage corresponding to the pressure applied to the capsule case 41. Based on the voltage generated in the piezoelectric sensor 44, the control circuit 42 recognizes the area where the capsule endoscope 40 exists.

The control circuit 42 is preliminary programmed to have a power saving mode which prevents the waste of the battery 19, and a normal capture mode which transmits the obtained image signal outwards. The control circuit 42 executes one of the power saving mode or the normal capture mode at one time. In the power saving mode, the power supply to the CCD image sensor 13, the LEDs 15 and the transmitting circuit 33 is stopped, and neither the image capturing nor the image transmission is performed.

The timer circuit 43 is activated when the capsule endoscope 40 is in use, and counts a certain period of time. Specifically, the timer circuit 43 counts presumed time from when the capsule endoscope 40 is swallowed till when the capsule endoscope 40 is in the small intestine. That is, the capsule endoscope 40 exists in the small intestine with extremely high probability when the timer circuit 43 finishes counting. The control circuit 42 monitors the pressure which the capsule case 41 receives since when the timer circuit 43 finishes counting. When the control circuit 42 detects that the pressure changes to that of the large intestine, the control circuit 42 switches from the power saving mode to the normal capture mode.

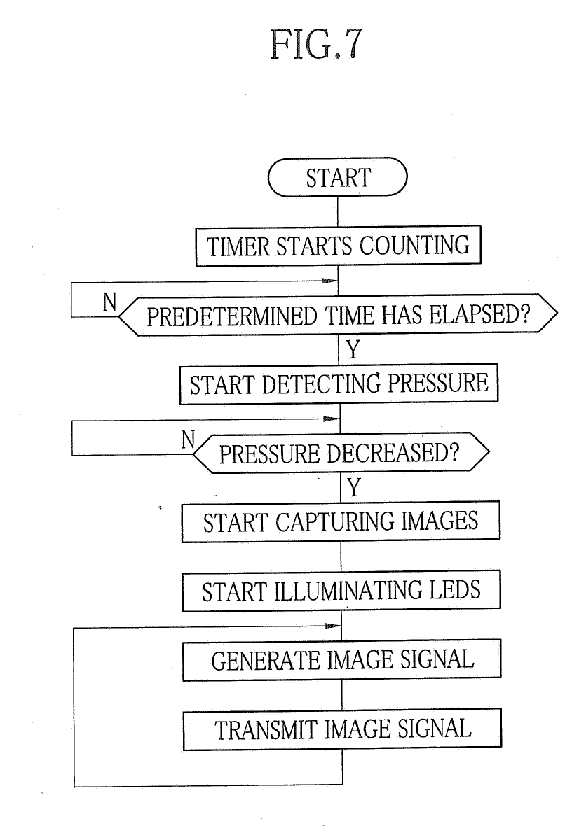

In FIG. 7, when the capsule endoscope 40 is swallowed by the patient, the control circuit 42 activates the timer circuit 43 to start counting the time. The control circuit 42 maintains the power saving mode and stands by until the timer circuit 42 finishes counting. In the power saving mode, the power supply to the CCD image sensor 13, the LEDs 15 and the transmitting circuit 33 is stopped.

When the timer circuit 43 finishes counting a certain period of time, the piezoelectric sensor 44 measures intensity of the pressure applied to the capsule case 41 from outside. The control circuit 42 memorizes the pressure of the first measurement as a standard value. After memorizing the standard value, the control circuit 42 starts monitoring the voltage generated in the piezoelectric sensor 44. In the small intestine, high pressure is applied to the capsule case 41. In the large intestine, on the other hand, low pressure is applied to the capsule case 41. The control circuit 42 compares the voltage generated in the piezoelectric sensor 44 with the standard value. When the pressure applied to the capsule case 41 becomes low, the control circuit 41 determines that the capsule endoscope 40 reaches the large intestine, and switches from the power saving mode to the normal capture mode.

The CCD image sensor 13 generates the image signals at the rate of 30 frames/sec. In accordance with that, the LEDs 15 illuminate 30 times/sec. The control circuit 42 transmits the image signals as the radio signals via the transmitting circuit 33. The external receiver 20 sends the received image signals to the image recording device 21. In this way, the captured images of the large intestine are stored in the image recording device 21.

Note that the present invention is not limited to the above embodiments. For example, the changeover from the power saving mode to the normal capture mode is performed only once in all of the above embodiments, however, the changeover between two operation modes can be performed more than once. Moreover, the present invention can be utilized in capturing images of the small intestine and the like.

Various changes and modifications are possible in the present invention and may be understood to be within the present invention.

What is claimed is:

1. A capsule endoscope including an image pickup section for capturing an image of a region to be inspected to generate an image signal, a light source section for emitting illumination light every time said image pickup section captures said image, an image transmitter for transmitting said image signal to an external receiver via radio waves, a battery for supplying electric power to each of said image pickup section, said light source section and said image transmitter, and a capsule case for containing said image pickup section, said light source section, said image transmitter and said battery, said capsule endoscope comprising:
an image capture controller having a power saving mode in which an image capturing rate per unit time is low and a normal capture mode in which said image capture rate per unit time is high, said image capture controller selecting one of said power saving mode and said normal capture mode to execute.

2. A capsule endoscope as claimed in claim 1, wherein said image capture controller selects said power saving mode in an initial state and selects said normal capture mode while said capsule endoscope moves inside of a body.

3. A capsule endoscope as claimed in claim 2, wherein said image capturing is not performed in said power saving mode.

4. A capsule endoscope as claimed in claim 2, further comprising a receiver for receiving a mode changeover signal from an external transmitter, wherein said image capture controller selects said normal capture mode when said receiver receives said mode changeover signal.

5. A capsule endoscope as claimed in claim 2, further comprising a pressure detecting section for detecting pressure from outside of said capsule case and a timer for counting time, wherein said image capture controller selects said normal capture mode when the pressure detected by said pressure detecting section changes from a standard value, said standard value being a pressure value at a time when said timer counts a predetermined amount of time.

6. A capsule endoscope as claimed in claim 2, further comprising an image analyzing section for analyzing a captured image in order to identify a predetermined region to be inspected, wherein said image capture controller selects said normal capture mode when said image analyzing section identifies said image captured in said power saving mode as an image of said predetermined region to be inspected.
